(19) United States
(12) Patent Application Publication
(10) Pub. No.: US 2009/0076283 A1
(43) Pub. Date: Mar. 19, 2009

(54) Method of crystallizing carvedilol phosphate and the product thereof

(75) Inventors: Wen-Li Shih, Shueili Township (TW); Shu-Ping Chen, Kaohsiung City (TW)

Correspondence Address: COHEN, PONTANI, LIEBERMAN & PAVANE LLP, 551 FIFTH AVENUE, SUITE 1210, NEW YORK, NY, 10176, US

(73) Assignee: ScinoPharm Taiwan Ltd., Tainan County (TW)

(21) Appl. No.: 12/231,760

(22) Filed: Sep. 5, 2008

Related U.S. Application Data
(60) Provisional application No. 60/967,934, filed on Sep. 7, 2007.

(51) Int. Cl. C07D 209/82

(52) U.S. Cl. 548/444

(57) ABSTRACT

The present invention provides for a crystalline polymorph of Carvedilol phosphate salt and a process for making the same.

### BACKGROUND OF THE INVENTION

1. Field of the Invention

The present application claims priority to Provisional Patent Application 60/967,934, filed Sep. 7, 2007. The contents of this provisional patent application is hereby incorporated by reference.

### SUMMARY OF THE INVENTION

The present invention provides a method of crystallizing Carvedilol-phosphate salt, and a novel crystalline polymorph of Carvedilol-phosphate salt.

### BRIEF DESCRIPTION OF THE DRAWINGS

In the drawings:

FIG. 1 shows the X-ray powder diffraction pattern of the crystalline polymorph of Carvedilol-phosphate salt of the present invention in intensity versus degrees in two theta.

FIG. 2 is the diffuse reflectance spectrum of the crystalline polymorph of Carvedilol-phosphate salt of the present invention.

FIG. 3 provides the peak positions within the diffuse reflectance spectrum of FIG. 2, as well as an expansion of a certain region of that spectrum.

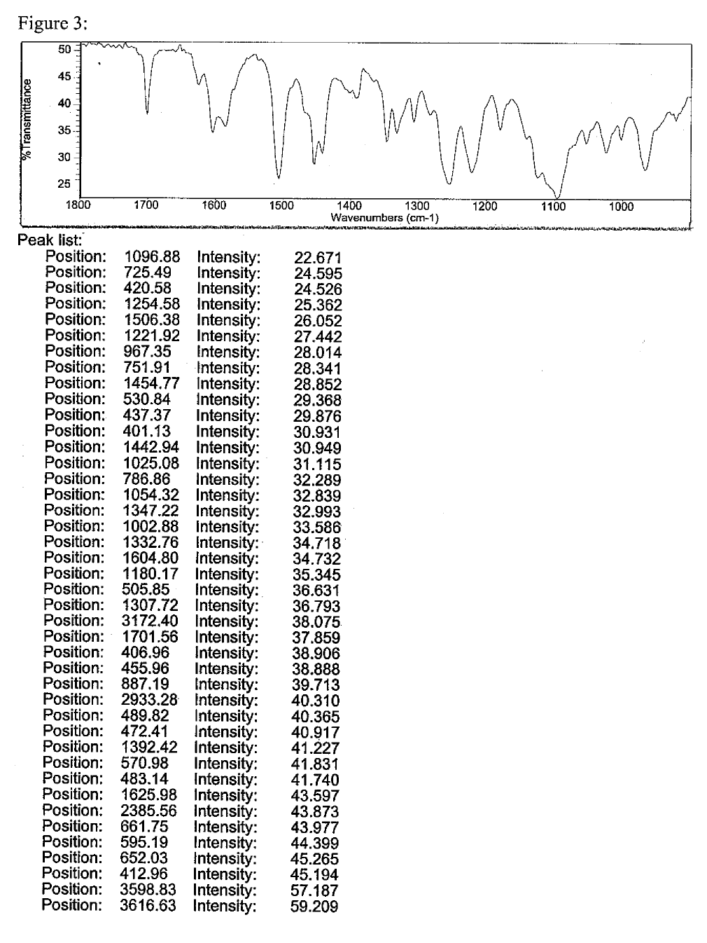

FIG. 4 shows the X-ray powder diffraction pattern of the crystalline polymorph of Carvedilol-phosphate salt of the present invention in intensity versus d-spacing.

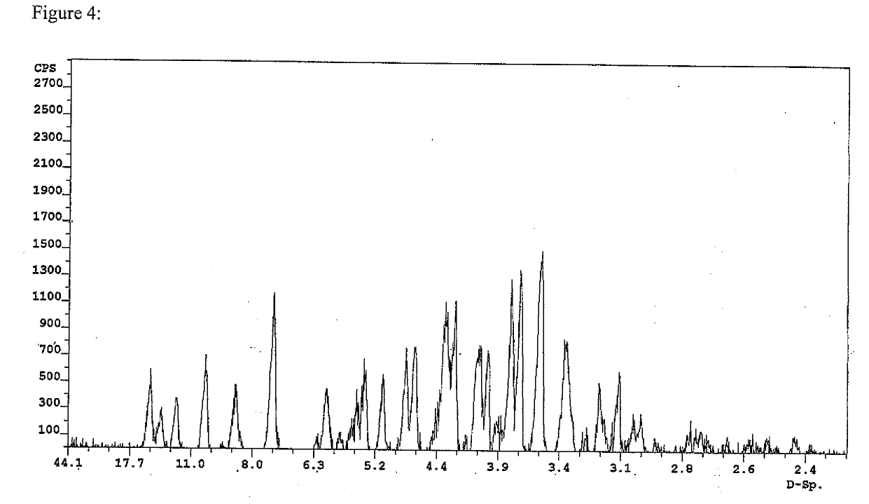

FIG. 5 lists the peak positions and their intensities of the X-ray powder diffraction pattern of the crystalline polymorph of Carvedilol-phosphate salt of the present invention.

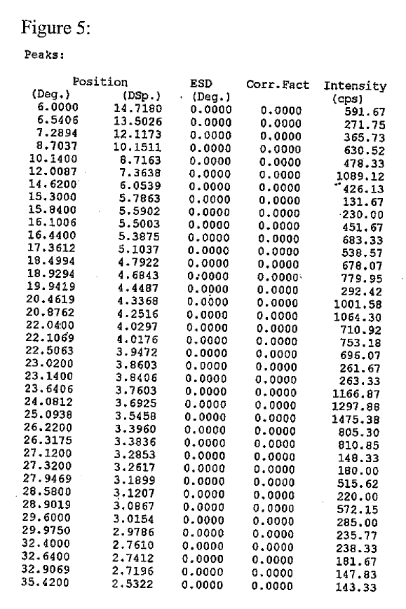

### DETAILED DESCRIPTION OF THE PRESENTLY PREFERRED EMBODIMENTS

The present invention provides a method of crystallizing Carvedilol-phosphate salt by the following procedure:

### Carvedilol Phosphate Salt Formation

To a solution of Carvedilol (5 g; 0.0123 mol.) in acetone (90 mL) is added 85% H₃PO₄ (0.85 g; 0.0074 mol.) slowly. The resulting mixture is agitated at 20-30° C. The mixture is filtered and then washed with 20 mL acetone. The solids are dried under vacuum at <50° C to give 4.76 g Carvedilol-phosphate salt (hemihydrate).

Representative infrared spectrum and X-ray powder diffraction pattern for the Carvedilol-phosphate salt is provided herein.

We claim:

1. A crystalline polymorph of Carvedilol-phosphate salt exhibiting an X-ray powder diffraction pattern comprising a peak in degrees 2θ±0.2°2θ at 25.1.

2. A crystalline polymorph of Carvedilol-phosphate salt according to claim 1 further comprising peaks in degrees 2θ±0.2°2θ at 24.1 and 23.6.

3. The crystalline polymorph of Carvedilol-phosphate salt according to claim 1 further comprising peaks in degrees 2θ±0.2°2θ at 12.0 and 20.9.

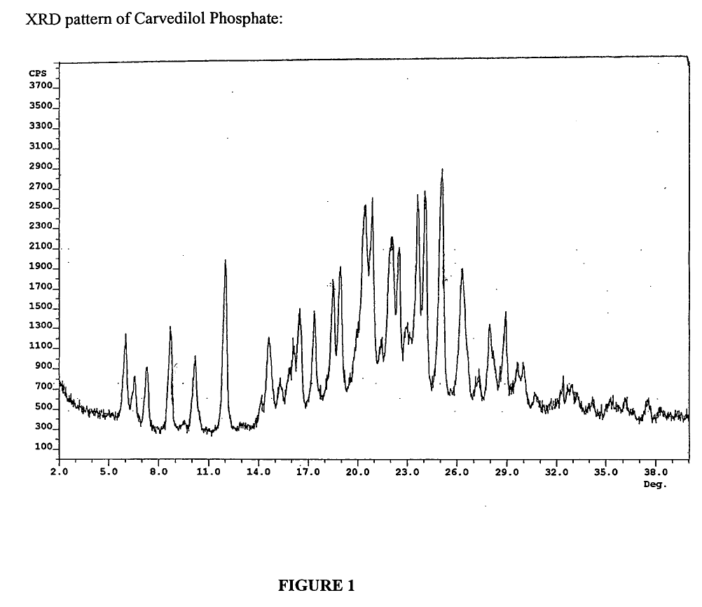

4. The crystalline polymorph of Carvedilol-phosphate salt according to claim 1 exhibiting an X-ray powder diffraction pattern substantially as shown in FIG. 1.

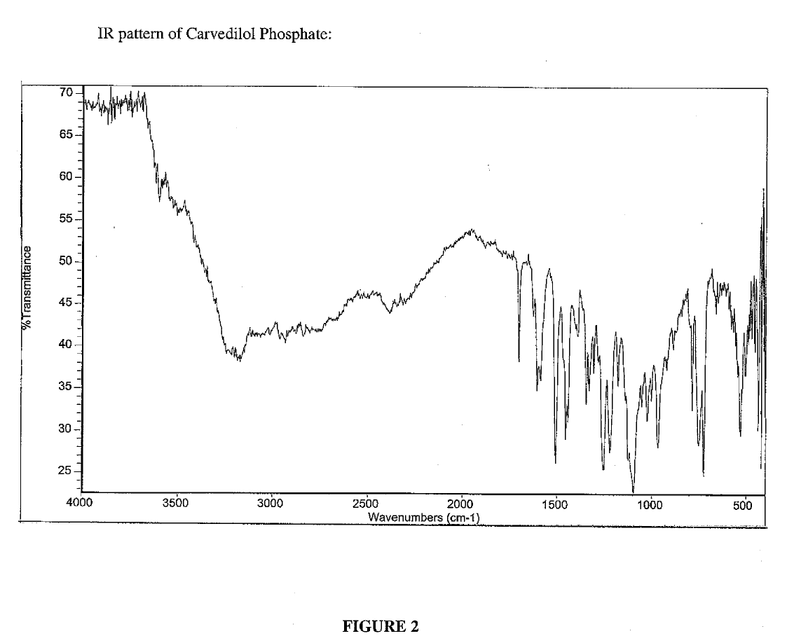

5. The crystalline polymorph of Carvedilol-phosphate salt according to claim 1 exhibiting an IR diffuse reflectance spectrum substantially as shown in FIG. 2.

6. A method of preparing a crystalline polymorph of Carvedilol-phosphate salt comprising the steps of: (a) dissolving Carvedilol in acetone to form a solution; and (b) adding a solution comprising H₃PO₄ to produce the crystalline polymorph of Carvedilol-phosphate salt.
